(19) United States
(12) Patent Application Publication
(10) Pub. No.: US 2023/0321296 A1
(43) Pub. Date: Oct. 12, 2023

(54) NOVEL CORONAVIRUS DISINFECTION AND STERILIZATION MOVABLE WORKSTATION FOR REFRIGERATED CONTAINER YARD AND METHOD USING MOVABLE WORKSTATION

(71) Applicant: Changhao WANG, Maoming City (CN)

(72) Inventors: Qun WANG, Maoming City (CN); Changhao WANG, Maoming City (CN); Gengyu CAO, Maoming City (CN)

(21) Appl. No.: 17/715,003

(22) Filed: Apr. 6, 2022

(51) Int. Cl. A61L 2/20

(52) U.S. Cl. CPC A61L 2/202; A61L 2202/14; A61L 2202/15; A61L 2202/23

(57) ABSTRACT

Disclosed are a novel coronavirus disinfection and sterilization movable workstation for a refrigerated container yard and a method using the movable workstation. The movable workstation comprises a moving device, a lifting device, a disinfection gas supply device, a vacuumizing device, a gas transmission pipeline, a gas inlet and outlet pipe orifice and a control system, wherein the lifting device and the disinfection gas supply device are arranged on the upper surface of the moving device; the lifting device comprises a lifting support and an operation table, and a manual operation fence is arranged on the operation table; the control system is arranged in the manual operation fence; the gas inlet and outlet pipe orifice is formed in the end of the gas transmission pipeline; and the disinfection gas supply device is connected with the gas transmission pipeline through a first branch pipeline.

### TECHNICAL FIELD

The present disclosure relates to the technical fields of cold chain transportation and disinfection, in particular to a novel coronavirus disinfection and sterilization movable workstation for a refrigerated container yard and a method using the movable workstation.

### BACKGROUND

For prevention and control of major infectious diseases, public health means are more important than medical means. The novel coronavirus also has a certain period of survival time outside the human body, and can be adsorbed on the surface of an object to cause contact transmission. Many cases show that positive novel coronavirus nucleic acids are detected on some cold chain food and/or cold chain food packages and/or cold chain food transportation tools. Cold chains can preserve food and also serve as routes for virus transmission, and once cold chain food and/or cold chain food packages and/or cold chain food transportation tools are contaminated with novel coronavirus during production, processing and circulation, the low-temperature and high-humidity environment of traditional cold chains is conducive to long-term vigor of the virus, which can cause epidemic situations. How to scientifically and effectively cut off the transmission path of novel coronavirus of cold chain food, cold chain food packages and cold chain food transportation tools and strengthen the supervision of cold chain food and all links in circulation is also important. The existing disinfection and sterilization method for novel coronavirus of the cold chain food has the defects that workers enter the container and carry the cold chain food to the conveyor belt, the conveyor belt conveys articles into the disinfection cabin, the articles are sprayed with disinfectant, the articles leave the disinfection cabin, the workers carry the cold chain food away from the conveyor belt for warehousing, the labor intensity of the workers is high, and the workers are prone to infecting viruses; and the outer surface of the package cannot be covered with the sprayed disinfectant completely, the sprayed disinfectant cannot enter the inner surface of the package, and the sprayed disinfectant cannot enter the surfaces of the articles in the packages. Therefore, there is a need for an effective disinfection device and method for cold chain logistics transportation, which cut off the paths of novel coronavirus transmission to cold chain food and/or cold chain food packages and/or cold chain food transportation tools.

### SUMMARY

The first technical problem to be solved by the present disclosure is to provide a novel coronavirus disinfection and sterilization movable workstation for a refrigerated container yard. The workstation is simple in device structure and easy to implement, can be conveniently moved near the refrigerated container yard needing to be disinfected, can effectively cut off new coronavirus transmission paths of cold chain food transportation tools, cold chain food therein and cold chain food package surfaces, and completely eradicates contact infection.

The second technical problem to be solved by the present disclosure is to provide a method using the movable workstation to kill novel coronavirus in a refrigerated container yard.

In order to solve the first technical problem, the present disclosure adopts the following technical scheme:

A novel coronavirus disinfection and sterilization movable workstation for a refrigerated container yard comprises a moving device, a lifting device, a disinfection gas supply device, a vacuumizing device, a gas transmission pipeline, a gas inlet and outlet pipe orifice and a control system, wherein
the lifting device and the disinfection gas supply device are arranged on the upper surface of the moving device;
the lifting device comprises a lifting support and an operation table, and a manual operation fence is arranged on the operation table; the control system is arranged in the manual operation fence;
the gas inlet and outlet pipe orifice is formed in the end of the gas transmission pipeline;
the disinfection gas supply device is connected with the gas transmission pipeline through a first branch pipeline;
the vacuumizing device is connected with the gas transmission pipeline through a second branch pipeline;
a valve is arranged on the gas transmission pipeline, or valves are arranged on the first branch pipeline and the second branch pipeline;
the control system is connected with the lifting device, the disinfection gas supply device and the vacuumizing device through data transmission lines; and the control system controls operation and closing of the lifting device, the disinfection gas supply device and the vacuumizing device.

In an embodiment, the movable workstation further comprises a coding device so as to record logistics information.

In an embodiment, the gas transmission pipeline is a telescopic pipeline; and the gas inlet and outlet pipe orifice is a pipeline connecting flange.

In an embodiment, the moving device is a vehicle.

In an embodiment, the vacuumizing system is a vacuumizing pump.

In an embodiment, the disinfection gas supply device comprises an ozone generator and an ozone pump.

In order to solve the second technical problem, the present disclosure adopts the following scheme:

A method using the movable workstation to kill novel coronavirus in a refrigerated container yard comprises the following steps:
S1, moving the novel coronavirus disinfection and sterilization movable workstation for a refrigerated container yard near the refrigerated container yard to be disinfected;
S2, lifting the movable workstation near a ventilation opening through a lifting device by workers, opening the ventilation opening of the refrigerated container by the workers, and connecting the gas inlet and outlet pipe orifice with the ventilation opening;
S3, opening a valve on a second branch pipeline, and closing a valve on a first branch pipeline, so that a vacuumizing device communicates with the refrigerated container;
S4, starting the vacuumizing device to vacuumize the refrigerated container;
S5, closing the vacuumizing system, and closing the valve on the second branch pipeline;
S6, opening the valve on the first branch pipeline so that the disinfection gas supply device communicates with the refrigerated container;
S7, starting the disinfection gas supply device to fill disinfection gas into the refrigerated container, so that gaps in the inner surface of the container, the contents of the container, namely cold chain materials, cold chain material packages and the inner wall of the refrigerated container are filled with the disinfection gas;
S8, enabling the pressure of the injected disinfection gas to be 0.8 to 1.2 atmospheric pressures;
S9, maintaining the disinfection gas in the refrigerated container for 3-3600 seconds;
S10, closing the valve on the first branch pipeline;
S11, separating the gas inlet and outlet pipe orifice from the ventilation opening of the refrigerated container, and closing the ventilation opening of the refrigerated container; and
S12, coding and recording disinfection operation.

Any range recorded herein includes the endpoints, any numerical value between the endpoints and any subranges subsumed by the endpoints or any numerical value between the endpoints.

If not specified, each raw material in the present disclosure is commercially available, and the equipment used in the present disclosure may be used by using conventional equipment in the art or by reference to the prior art in the art.

Compared with the prior art, the present disclosure has the following beneficial effects:
firstly, the movable workstation can effectively cut off the new coronavirus transmission paths of cold chain food transportation tools, cold chain food therein and cold chain food package surfaces;
secondly, the disinfected transportation tools are coded, so that the traceability is realized;
thirdly, after the ozone used in the movable workstation is reacted, high-concentration oxygen can keep the freshness of cold chain materials;
fourthly, by utilizing the characteristics of gas diffusion and permeation, the holes and gaps of corrugated paper packaging boxes are utilized to enable disinfection gas to be in contact with the outer surface of the package, the packaging material, the inner surface of the package and the surface of articles in the package to kill viruses, so that the carrying operation of workers is avoided, the workers do not need to enter the container, the workers do not need to be in contact with cold chain materials, and the infection of the coronavirus is avoided;
fifthly, the movable workstation can be conveniently moved near the refrigerated container for operation, is suitable for a refrigerated container yard environment and is also suitable for a refrigerated container transportation vehicle parking area, and a special place does not need to be occupied;
sixthly, other closed refrigerated spaces can also be disinfected by using the movable workstation; and
seventhly, workers can work on the operation table, so that the workstation is conveniently controlled to operate, the ventilation opening of the refrigerated container is conveniently opened, the gas inlet and outlet pipe orifice and the ventilation opening of the refrigerated container are conveniently combined and separated, and the ventilation opening of the refrigerated container is conveniently closed.

In short, the movable workstation can effectively cut off new coronavirus transmission paths of cold chain food transportation tools, cold chain food therein and cold chain food package surfaces, and the workers are avoided from being in contact with cold chain materials, so that contact infection is completely eradicated.

### BRIEF DESCRIPTION OF THE DRAWINGS

The following further describes specific embodiments of the present disclosure with reference to the following attached figures.

FIG. 1 is a structural schematic diagram of an embodiment of a movable workstation in the present disclosure;

FIG. 2 is an application state diagram of an embodiment of a novel coronavirus disinfection and sterilization movable workstation for a refrigerated container yard in the present disclosure; and

FIG. 3 is a control box diagram of an embodiment of a control system in the present disclosure.

### DETAILED DESCRIPTION OF THE EMBODIMENTS

In order to describe the present disclosure more clearly, the present disclosure is further described in detail in conjunction with the attached figures. Those skilled in the art will appreciate that what is specifically described below is illustrative and not restrictive, and should not limit the scope of the present disclosure.

It should be noted that when one element is referred to as being “fixed” or “arranged” on the other element, it can be directly on the other element or indirectly on the other element. When one element is referred to as being “connected” to the other element, it may be directly connected to the other element or indirectly connected to the other element.

The indicative direction or position relations of the terms such as “length”, “width”, “upper”, “lower”, “front”, “back”, “left”, “right”, “vertical”, “horizontal”, “top”, “bottom”, “inside” and “outside” are direction or position relations illustrated based on the accompanying diagrams, just for facilitating the description of the present disclosure and simplifying the description, but not for indicating or hinting that the indicated device or element must be in a specific direction and is constructed and operated in the specific direction, the terms cannot be understood as the restriction of the present disclosure.

In addition, the terms “first” and “second” are merely intended for a purpose of description, and shall not be understood as an indication or implication of relative importance or implicit indication of the number of indicated technical features. Therefore, a feature limited by “first” or “second” may include one or more features explicitly or implicitly. In the description of the present disclosure, the meaning of “a plurality of” means two or more unless expressly specifically defined otherwise.

### Embodiment I

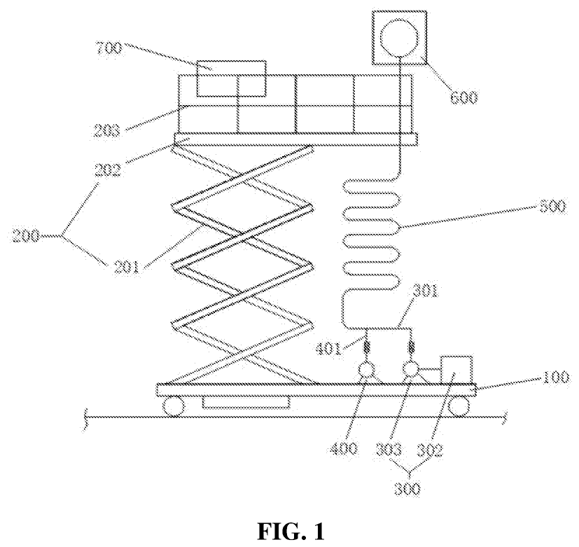

As shown in FIG. 1, a novel coronavirus disinfection and sterilization movable workstation for a refrigerated container yard comprises a moving device 100, a lifting device 200, a disinfection gas supply device 300, a vacuumizing device 400, a gas transmission pipeline 500, a gas inlet and outlet pipe orifice 600 and a control system 700, wherein
the lifting device 200 and the disinfection gas supply device 300 are arranged on the upper surface of the moving device 100;
the lifting device 200 comprises a lifting support 201 and an operation table 202, and a manual operation fence 203 is arranged on the operation table 202; the control system 700 is arranged in the manual operation fence 203; it is understandable that by means of the lifting device 200, an operator can operate the control system 700 in the manual operation fence 203 and adjust the height and horizontal position of the operation table 202 to accommodate refrigerated containers 800 at different positions within the yard 900;
the gas inlet and outlet pipe orifice 600 is formed in the end of the gas transmission pipeline 500;
the disinfection gas supply device 300 is connected with the gas transmission pipeline 500 through a first branch pipeline 301;
the vacuumizing device 400 is connected with the gas transmission pipeline 500 through a second branch pipeline 401; it is understandable that, as a variation of the present disclosure, the first branch pipe 301 and the second branch pipe 401 cannot be merged in the gas transmission pipe, but is separately provided with a gas inlet and outlet pipe orifice connected with the ventilation opening of the refrigerated container, and this variation is also within the scope of protection of the present disclosure;

Valves are arranged on the first branch pipe 301 and the second branch pipe 401;

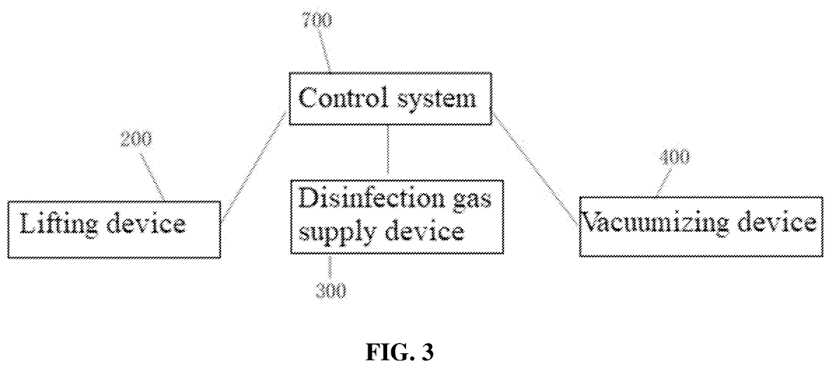

As shown in FIG. 3, the control system 700 is connected with the lifting device 200, the disinfection gas supply device 300 and the vacuumizing device 400 through data transmission lines; and the control system 700 controls operation and closing of the lifting device 200, the disinfection gas supply device 300 and the vacuumizing device 400.

In the embodiment, the movable workstation further comprises a coding device (unshown in the figures) so as to record logistics information.

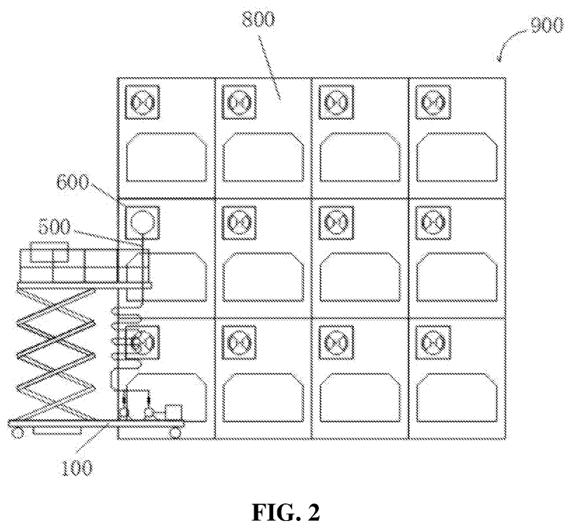

As shown in FIG. 2, in the embodiment, the gas transmission pipeline 500 is a telescopic pipeline; and the gas inlet and outlet pipe orifice 600 is a pipeline connecting flange. In the using process, the gas inlet and outlet pipe orifice 600 is in butt joint with the ventilation opening of the refrigerated container 800, the refrigerated container is vacuumized, and disinfection gas is injected into the refrigerated container.

In the embodiment, the moving device 100 is a vehicle.

In the embodiment, the vacuumizing system 400 is a vacuumizing pump.

In the embodiment, the disinfection gas supply device 300 comprises an ozone generator 302 and an ozone pump 303.

### Embodiment II

A method using the movable workstation to kill novel coronavirus on refrigerated containers comprises the following steps:
S1, moving the movable workstation capable of killing novel coronavirus in a refrigerated container yard near the refrigerated container yard to be disinfected;
S2, lifting the movable workstation near a ventilation opening through a lifting device by a worker, opening the ventilation opening of the refrigerated container by the worker, and connecting the gas inlet and outlet pipe orifice of a gas transmission device with the ventilation opening;
S3, opening a valve on a second branch pipeline, and closing a valve on a first branch pipeline, so that a vacuumizing device communicates with the refrigerated container;
S4, starting the vacuumizing device to vacuumize the refrigerated container;
S5, closing the vacuumizing system, and closing the valve on the second branch pipeline;
S6, opening the valve on the first branch pipeline so that the disinfection gas supply device communicates with the refrigerated container;
S7, starting the disinfection gas supply device to fill disinfection gas into the refrigerated container, so that gaps in the inner surface of the container, the contents of the container, namely cold chain materials, cold chain material packages and the inner wall of the refrigerated container are filled with the disinfection gas;
S8, enabling the pressure of the injected disinfection gas to be 0.8 to 1.2 atmospheric pressures;
S9, maintaining the disinfection gas in the refrigerated container for 3-3600 seconds;
S10, closing the valve on the first branch pipeline;
S11, separating the gas inlet and outlet pipe orifice of the gas transmission device from the ventilation opening of the refrigerated container, and closing the ventilation opening of the refrigerated container; and
S12, coding and recording disinfection operation.

In conclusion, the movable workstation can effectively kill novel coronavirus in the cold chain food, the cold chain food package and the cold chain food packaging boxes; a vibration device or a container vehicle is arranged to jolt on a road surface, so that gaps are formed among the containers, articles in the container and packaging boxes of the articles in the container due to vibration, ozone can be easily diffused in the vibration gaps, and viruses on the surfaces of the packaging boxes of the articles in the container are efficiently eliminated and killed. According to the movable workstation, the disinfected vehicles are coded, so that the traceability is realized. After the ozone used in the movable workstation is reacted, high-concentration oxygen can keep the freshness of cold chain materials.

Obviously, the embodiments of the present disclosure are merely illustrative of the present disclosure as examples and are not intended to be limitation of embodiments of the present disclosure. For those of ordinary skill in the art, other variations or modifications in different forms may be made on the basis of the above description. All embodiments are impossible to be exhaustive herein. Obvious changes or variations which belong to the technical scheme of the present disclosure are still within the scope of protection of the present disclosure.

What is claimed is:

1. A novel coronavirus disinfection and sterilization movable workstation for a refrigerated container yard, comprising a moving device, a lifting device, a disinfection gas supply device, a vacuumizing device, a gas transmission pipeline, a gas inlet and outlet pipe orifice and a control system, wherein
the lifting device is arranged on the upper surface of the moving device;
the lifting device comprises a lifting support and an operation table, and a manual operation fence is arranged on the operation table; the control system is arranged in the manual operation fence;
the gas inlet and outlet pipe orifice is formed in the end of the gas transmission pipeline;
the disinfection gas supply device is connected with the gas transmission pipeline through a first branch pipeline;
the vacuumizing device is connected with the gas transmission pipeline through a second branch pipeline;
a valve is arranged on the gas transmission pipeline, or valves are arranged on the first branch pipeline and the second branch pipeline;
the control system is connected with the lifting device, the disinfection gas supply device and the vacuumizing device through data transmission lines; and the control system controls operation and closing of the lifting device, the disinfection gas supply device and the vacuumizing device.

2. The movable workstation according to claim 1, wherein the movable workstation further comprises a coding device.

3. The movable workstation according to claim 1, wherein the gas transmission pipeline is a telescopic pipeline; and the gas inlet and outlet pipe orifice is a pipeline connecting flange.

4. The movable workstation according to claim 1, wherein the moving device is a vehicle.

5. The movable workstation according to claim 1, wherein the vacuumizing system is a vacuumizing pump.

6. The movable workstation according to claim 1, wherein the disinfection gas supply device comprises an ozone generator and an ozone pump.

7. A method using the movable workstation to kill novel coronavirus in a refrigerated container yard according to claim 1, comprising the following steps:
S1, moving the novel coronavirus disinfection and sterilization movable workstation for a refrigerated container yard near the refrigerated container yard to be disinfected;
S2, lifting the movable workstation near a ventilation opening through a lifting device by workers, opening the ventilation opening of the refrigerated container by the workers, and connecting the gas inlet and outlet pipe orifice with the ventilation opening;
S3, opening a valve on a second branch pipeline, and closing a valve on a first branch pipeline, so that a vacuumizing device communicates with the refrigerated container;
S4, starting the vacuumizing device to vacuumize the refrigerated container;
S5, closing the vacuumizing system, and closing the valve on the second branch pipeline;
S6, opening the valve on the first branch pipeline so that a disinfection gas supply device communicates with the refrigerated container;
S7, starting the disinfection gas supply device to fill disinfection gas into the refrigerated container, so that gaps in the inner surface of the container, the contents of the container, namely cold chain materials, cold chain material packages and the inner wall of the refrigerated container are filled with the disinfection gas;
S8, enabling the pressure of the injected disinfection gas to be 0.8 to 1.2 atmospheric pressures;
S9, maintaining the disinfection gas in the refrigerated container for 3-3600 seconds;
S10, closing the valve on the first branch pipeline;
S11, separating the gas inlet and outlet pipe orifice from the ventilation opening of the refrigerated container, and closing the ventilation opening of the refrigerated container; and
S12, coding and recording disinfection operation.

8. The method according to claim 7, wherein the movable workstation further comprises a coding device.

9. The method according to claim 7, wherein the gas transmission pipeline is a telescopic pipeline; and the gas inlet and outlet pipe orifice is a pipeline connecting flange.

10. The method according to claim 7, wherein the moving device is a vehicle.

11. The method according to claim 7, wherein the vacuumizing system is a vacuumizing pump.

12. The method according to claim 7, wherein the disinfection gas supply device comprises an ozone generator and an ozone pump.
